(19) United States
(12) Patent Application Publication
(10) Pub. No.: US 2018/0132781 A1
(43) Pub. Date: May 17, 2018

(54) Artery Locating Device and Automated Sampling Device for Arterial Blood Gas Measurement

(71) Applicant: Homayemem Weli-Numbere, Coventry (GB)

(72) Inventor: Homayemem Weli-Numbere, Coventry (GB)

(21) Appl. No.: 15/813,141

(22) Filed: Nov. 14, 2017

Related U.S. Application Data
(60) Provisional application No. 62/422,186, filed on Nov. 15, 2016.

(51) Int. Cl. A61B 5/00; A61B 5/145; A61B 5/153

(52) U.S. Cl. CPC A61B 5/489; A61B 5/153; A61B 5/14542; A61B 5/0082

(57) ABSTRACT

Method and device for using energy transduction for novel use in locating an underlying artery, particularly a more superficial artery like the radial artery. The method includes Placing or scanning the device containing an electrical system, which comprises of an arterial pressure or motion transducer converted to visible light energy on the overlying skin in the region of the vessel. Then, observing for visibly emitted light to indicate the presence of an underlying artery, and localizing artery with a device, and then stabilizing the underlying blood vessel with the device placed on the overlying skin prior to introduction of arterial line or syringe. The device may include an automated sampling compartment for syringe introduction in the case of arterial blood gas collection.

### CROSS-REFERENCE TO RELATED APPLICATIONS

The present Utility patent application claims priority benefit of the U.S. provisional application for patent Ser. No. 62/422,186 entitled “Artery Locating Device and Automated Sampling Device for Arterial Blood Gas Measurement” filed on 15 Nov. 2016 under 35 U.S.C. 119(e). The contents of this related provisional application are incorporated herein by reference for all purposes to the extent that such subject matter is not inconsistent herewith or limiting hereof.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### FIELD OF THE INVENTION

The invention relates generally to an artery locating for arterial blood gas sampling.

### BACKGROUND

In the practice of medicine an arterial blood gas (“ABG”) test is common diagnostic tool utilized in the treatment of patients. Currently, to gather the blood required for testing, an artery is punctured with a thin needle and syringe where it is collected and transferred to the testing instrument. Often, it is difficult to locate the artery for sampling. Further, in the course of attempting to locate the artery, patients may be subjected to repeated unsuccessful needle punctures or even inadvertent venous punctures. Therefore, there is a need to a device to aid in the location and sampling of arterial blood for ABG testing.

### SUMMARY OF THE INVENTION

It would be advantageous to have an apparatus that improves the process of gathering an arterial blood gas sample. Furthermore, it would also be advantageous to have an apparatus that has a detachable or separate artery-locating unit. Still further, it would be advantageous to have an apparatus that improves and/or enables patient selfmonitoring in conditions like chronic obstructive pulmonary disease. Therefore, there currently exists a need in the market for an apparatus that merges precise artery detection and stabilization with automated sampling to ensure precision and sensitivity.

The invention advantageously fills the aforementioned deficiencies by providing an artery locator for arterial blood gas sampling, which limits user errors and can significantly improve or enable patient self-monitoring in conditions like chronic obstructive pulmonary disease.

The invention includes the use a motion sensor (a mini ultrasonic device), pressure sensor or biosensor transducers to signal by visible light to an operator the presence of an artery. This system is contained in a compartment next to a slot for the appropriate blood gas syringe. This syringe holder is connected to a lever system that can be moved precisely to inject the needle to the depth of the artery underneath the skin. It is important to note that the pressure/motion sensor tip which contacts the skin would be directly in parallel to the syringe tip such that it doubles as an artery stabilizer while he operator automatically triggers syringe descent to initiate blood sampling. The apparatus may comprise both an artery locator/detector unit and automated sampler unit or an artery-locating unit without the sampler unit. Ideally both units would complement each other.

The apparatus has components for specific identification of an artery and stabilization of the same artery prior to sampling. It merges precise artery detection and stabilization with automated sampling. It ensures precision and sensitivity.

The apparatus fulfills the need for a detachable or separate artery-locating unit, which is preferably handheld, similar to a pen or pen-like in configuration. It would have parallel sampling and detecting units to enable subsequent user/triggered automated sampling. Among other things, it is an advantage of the invention to provide an artery locator for arterial blood gas sampling that does not suffer from any of the problems or deficiencies associated with prior solutions.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 shows a combination sampling and locating device, according to the present invention;

FIG. 2 shows a locating device, according to the present invention;

FIG. 3 shows an alternate combination sampling and locating device, according to the present invention; and

FIG. 4 shows an alternate locating device, according to the present invention.

### DETAILED DESCRIPTION OF THE INVENTION

Following are more detailed descriptions of various concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

The invention is directed to be an artery locator for arterial blood gas sampling. The invention is a locator tip and a motion sensor (a mini ultrasonic device) and biosensor (oxygen sensor) to signal to an operator the presence of an artery. An energy/battery compartment with the option of connection to power source through a cord is utilized to power the device. The locator/detector unit is contained in a compartment next to a slot for the appropriate blood gas syringe. This syringe holder is connected to a lever system that can be moved precisely to inject the needle to the depth of the artery underneath the skin. It is important to note that the motion sensor tip which contacts the skin would be directly in parallel to the syringe tip such that it doubles as an artery stabilizer while the operator automatically triggers syringe descent to initiate blood sampling. A simplified locator detector unit can comprise a non-pricking indenter or marker tip that can be handheld and have disposable caps for single usage. This hand held unit can be used independently of the automated sampler system. When designed for handheld use, it is preferred that the device utilizes batteries for portability.

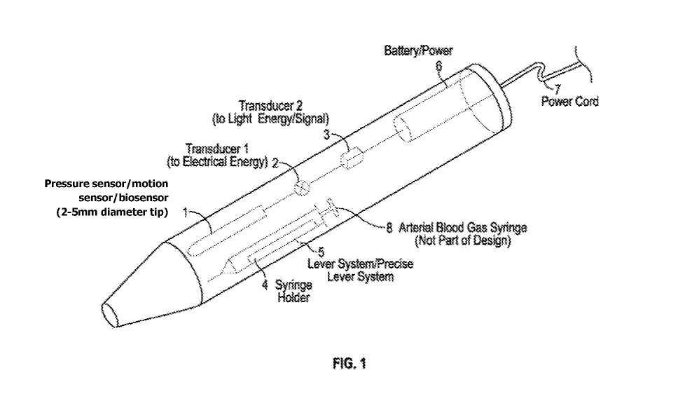

Referring to the figures, FIGS. 1-4 show multiple views and embodiments of an artery locating and sampling device. In FIG. 1, the device is generally pen shaped or penlike having a tip 1. The tip 1 is generally tapered and conical in shape and defining a skin contacting surface at a diameter between 2 to 5 millimeters (2-5 mm). The tip 1 includes a motion/biosensor capable of recognizes the location of an artery when placed upon the skin surface of a subject. Accordingly, this biosensor is coupled to a transducer 2 to convert a positive sensing to a light signal 3, wherein the user is altered to the presence of an artery through the light signal 3. The device includes a syringe holder 4 and lever system 5 for activation of a standard arterial blood gas collection syringe 8. The device includes a battery 6 and alternately a power cord 7 for providing power to the device.

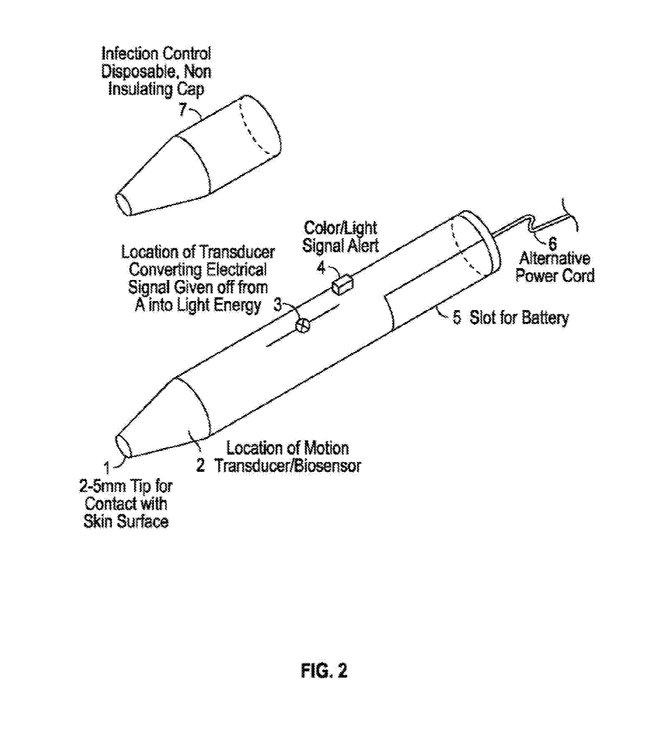

Referring now to FIG. 2, the device is utilized to locate an artery and does not include a sampling component, such as a syringe. The device utilizes a tip 1, biosensor 2, transducer 3, and signal 4, wherein the tip 1 is placed upon the skin of a subject for locating an artery with the biosensor 2 and transducing 3 a detected artery to an electric signal for powering a light/signal 4. The device includes a compartment for a battery 5 and 6 an alternate power cord 6. The device may be utilized with a disposable cap 7 to prevent the transmission of fluids between patients.

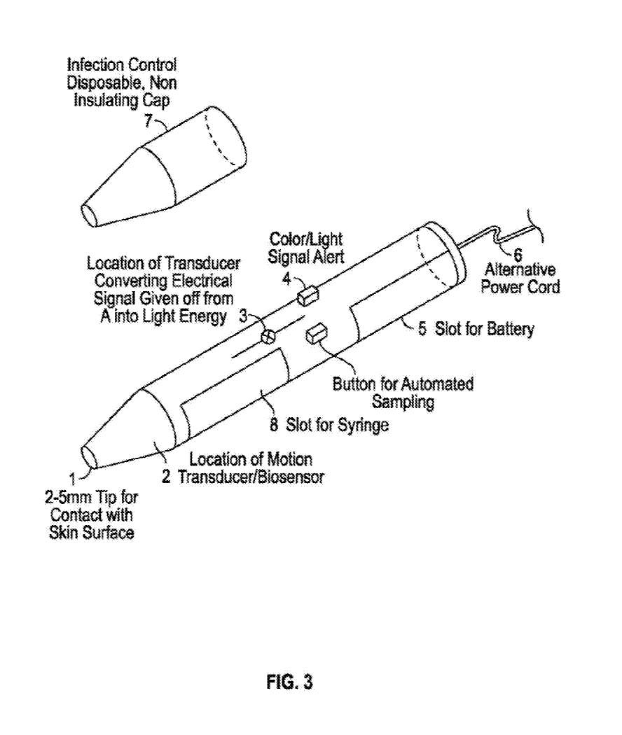

Referring now to FIG. 3, the device of FIG. 2 is provided with a slot or cavity 8 for the placement of a syringe for the automated sampling of arterial blood.

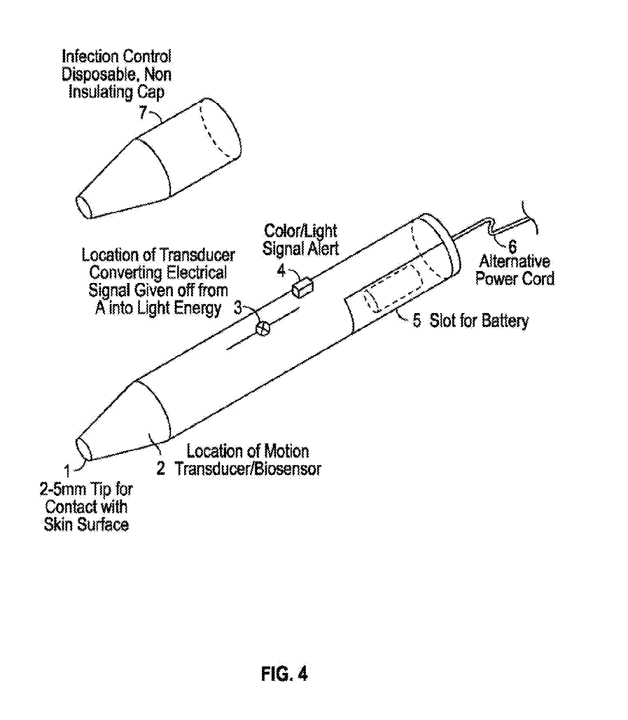

Referring now to FIG. 4, the device of FIG. 2 is pictured with a battery in the battery compartment for portable use. While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. An artery locating and sampling device, comprising:
pressure transducer or motion transducer means adapted to emit visible light and achieve the identification of underlying blood vessel (artery) for arterial line insertion or arterial blood gas sampling; and,
means for an automated sampling syringe compartment for arterial blood gas sampling.

2. The unique adaptation of a pressure transducer or motion transducer translated to visible light in the identification of underlying blood vessel (artery) for arterial line insertion or arterial blood gas sampling

3. The concept of a portable handheld light emitting device as described in 1 above (including but not limited to a “pen-like” form) for locating an underlying artery

4. A parallel system of an artery locator and automated sampling syringe compartment for arterial blood gas sampling
